(19) United States
(12) Patent Application Publication
(10) Pub. No.: US 2002/0025943 A1
(43) Pub. Date: Feb. 28, 2002

(54) Nucleoside analog compositions and uses thereof

(76) Inventors: Matthews O. Bradley, Laytonsville, MD (US); Charles S. Swindell, Merion, PA (US); Nigel L. Webb, Bryn Mawr, PA (US); Victor Shashoua, Belmont, MA (US)

Correspondence Address: Edward R. Gates, Esq., Wolf, Greenfield & Sacks, P.C., 600 Atlantic Avenue, Boston, MA, 02210, US

(21) Appl. No.: 09/948,472

(22) Filed: Sep. 6, 2001

Related U.S. Application Data
(63) Continuation of application No. 08/978,114, filed on Nov. 25, 1997.

(51) Int. Cl. A61K 31/7068

(52) U.S. Cl. 514/49

(57) ABSTRACT

The invention provides compositions that include conjugates of a carrier molecule, preferably cis-docosahexaenoic acid, and 2′,3′-dideoxycytidine. The conjugates are useful in treating viral infections, especially retroviral infections, and particularly reservoirs of viral infection in peripheral T cells and central nervous system manifestations thereof.

### RELATED APPLICATIONS

This application claims priority under 35 U.S.C. §120 from U.S. patent application Ser. Nos. 08/651,428, and 08/651,312, both filed May 22, 1996 the entire disclosures of which are incorporated herein by reference.

### BACKGROUND OF THE INVENTION

Dideoxycytidine (ddC) is an antiretroviral agent, administered orally, that inhibits the human immunodeficiency virus (HIV) from replicating. ddC is a nucleoside analog (like AZT and ddI), which inhibits the action of reverse transcriptase, an HIV enzyme that is critical in the replication of new virus. While ddC works by a mechanism similar to AZT, it has a different toxicity profile and persons who cannot tolerate the side effects of AZT may better tolerate ddC. ddC initially was approved for use in combination with AZT for persons with fewer than 300 CD4⁺ T cells. ddC has also been approved as monotherapy treatment of HIV for people with advanced HIV disease who either have experienced disease progression or are intolerant to AZT.

Similar to other nucleoside analogs, ddC has a number of side effects which are usually temporary and typically resolve within two weeks of initiating therapy. The “temporary” side effects include rashes, chest pain, fever, nausea, elevated liver enzymes and mouth sores.

The most serious side effect of ddC is dose-related nerve damage, called peripheral neuropathy, typically characterized by sharp burning pain sensations in the feet, legs, and/or hands. Severe neuropathy has been documented in patients at high doses of ddC (see, e.g., Simpson et al., J. Acquir. Immune Defic. Syndr. Hum. Retrovirol. 9(2):153-161, 1995).

Another serious side effect of ddC is pancreatitis, which is characterized by a sharp pain in the upper abdomen and nausea and vomiting. Incidence of pancreatitis seems to correlate to the higher dose and further advanced stages of illness. While pancreatitis is a serious side effect of ddC, incidence of pancreatitis associated with ddC use is lower than for persons taking ddI. Other rare but serious side effects of ddC include esophageal ulcers and congestive heart failure.

Fatty acids previously have been conjugated with drugs to help the drugs as conjugates cross the blood brain barrier. DHA (docosahexaenoic acid) is a 22 carbon naturally-occurring, unbranched fatty acid that previously has been shown to be unusually effective in crossing the blood brain barrier. When DHA is conjugated to a drug, the entire drug-DHA conjugate is transported across the blood brain barrier and into the brain.

DHA is attached via the acid group to hydrophilic drugs and renders these drugs more hydrophobic (lipophilic). DHA is an important constituent of the brain and recently has been approved as an additive to infant formula. It is present in the mink of lactating women. The mechanism of action by which DHA helps drugs conjugated to it cross the blood brain barrier is unknown.

Another example of the conjugation of fatty acids to a drug is the attachment of pipotiazine to stearic acid, palmitic acid, enanthic acid, undecylenic acid or 2,2-dimethyl-palmitic acid. Pipotiazine is a drug that acts within the central nervous system. The purpose of conjugating pipotiazine to the fatty acids was to create an oily solution of the drug as a liquid implant for slow release of the drug when injected intramuscularly. The release of the drug appeared to depend on the particular fatty acid selected, and the drug was tested for its activity in the central nervous system.

Lipidic molecules, including the fatty acids, also have been conjugated with drugs to render the conjugates more lipophilic than the drug. In general, increased lipophilicity has been suggested as a mechanism for enhancing intestinal uptake of drugs into the lymphatic system, thereby enhancing the entry of the conjugate into the brain and also thereby avoiding first-pass metabolism of the conjugate in the liver. The type of lipidic molecules employed have included phospholipids, non-naturally occurring branched and unbranched fatty acids, and naturally occurring branched and unbranched fatty acids ranging from as few as 4 carbon atoms to more than 30 carbon atoms. In one instance, enhanced receptor binding activity was observed (for an adenosine receptor agonist), and it was postulated that the pendant lipid molecule interacted with the phospholipid membrane to act as a distal anchor for the receptor ligand in the membrane micro environment of the receptor. This increase in potency, however, was not observed when the same lipid derivatives of adenosine receptor antagonists were used, and generalizations thus were not made possible by those studies.

### SUMMARY OF THE INVENTION

It has now been discovered that covalent conjugates of a fatty acid such as DHA and dideoxycytidine (ddC) have the unexpected property of increased potency against human immunodeficiency virus (HIV) as compared to unconjugated ddC. The level of increase is entirely unexpected and unpredictable. Potency was increased by a factor of 50. The ddC-fatty acid conjugate thus can be administered at lower molar doses which have increased antiviral activity yet are less likely to cause side effects typical of ddC treatment.

According to one aspect of the invention, a composition of matter is provided. The composition of matter is a covalent conjugate of 2′,3′-dideoxycytidine and a first fatty acid having 12-26 carbons, wherein the 2′,3′-dideoxycytidine has a nitrogen at the 4-carbon of a pyrimidine ring and has a pentose ring and wherein the fatty acid is conjugated to the nitrogen at the 4-carbon of the pyrimidine ring. It is preferred that the fatty acid is an unbranched, naturally occurring fatty acid. More preferably, the fatty acid has 14-22 carbons. Still another preferred embodiment comprises the fatty acid conjugated to 2′,3′-dideoxycytidine via an amide bond between the COOH of the first fatty acid and the NH at the 4-carbon of the pyrimidine ring.

Unbranched, naturally occurring fatty acids include C12:0 (lauric acid), C14:0 (myristic acid), C16:0 (palmitic acid), C16:1 (palmitoleic acid), C16:2, C18:0 (stearic acid), C18:1 (oleic acid), C18:1-7 (vaccenic), C18:2-6 (linoleic acid), C18:3-3 (α-linolenic acid), C18:3-5 (eleostearic), C18:3-6 (β-linolenic acid), C18:4-3, C20:1 (gondoic acid), C20:2-6, C20:3-6 (dihomo-y-linolenic acid), C20:4-3, C20:4-6 (arachidonic acid), C20:5-3 (eicosapentaenoic acid), C22:1 (docosenoic acid), C22:4-6 (docosatetraenoic acid), C22:5-6 (docosapentaenoic acid), C22:5-3 (docosapentaenoic), C22:6-3 (docosahexaenoic acid) and C24:1-9 (nervonic). Highly preferred unbranched, naturally occurring fatty acids are those with between 14 and 22 carbon atoms. Most preferred is docosahexaenoic acid.

The most preferred covalent conjugate is

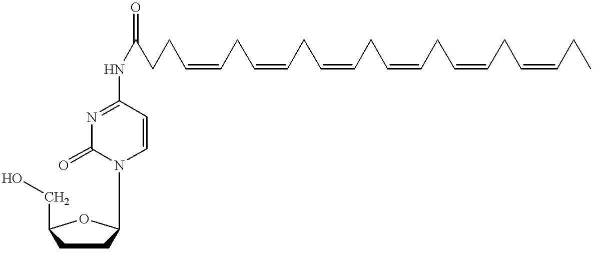

Another preferred conjugate, the less preferred than the foregoing conjugate, any of the conjugates described above further comprising a second fatty acid conjugated to the pentose ring. Preferred second fatty acids are as described above with respect to the first fatty acid. The preferred bond comprises the second fatty acid conjugated to the 2′,3′-dideoxycytidine via an ester bond between the COOH of the fatty acid and the pentose ring. The most preferred molecule having a second fatty acid is

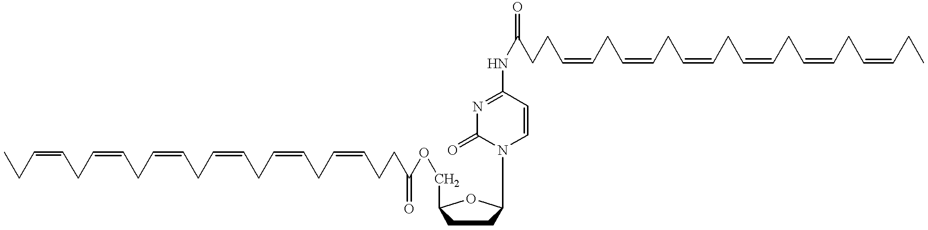

According to another aspect of the invention, pharmaceutical compositions are provided. The pharmaceutical compositions comprise any one of the covalent conjugates described above in an amount effective for treating a viral infection and the pharmaceutically acceptable carrier. Preferred conjugates are as described above. The pharmaceutical compositions also may further comprise an antiviral agent other than the covalent conjugate, such as a cocktail of antiviral compositions. Preferred such antiviral agents are selected from the group consisting of nucleoside analogs, non-nucleoside reverse transcriptase inhibitors, protease inhibitors and integrase inhibitors.

According to another aspect of the invention, a kit is provided. The kit comprises a package which houses a container containing the covalent conjugate as described above and also houses instructions for administering the covalent conjugate a subject having a viral infection.

According to another aspect of the invention, a second kit is provided. This kit comprises a package which houses a first container containing the covalent conjugate described above and which also houses a second container containing an antiviral agent other than the covalent conjugate.

In the foregoing kits, preferred fatty acids, bonds, covalent conjugates and antiviral agents other than the covalent conjugates are as described above.

According to another aspect of the invention, a method is provided for treating an non-brain viral infection. The method involves administering to a subject in need of such treatment an amount of a covalent conjugate of 2′,3′-dideoxycytidine and a first fatty acid having 12-26 carbons effective to treat the viral infection. Preferred fatty acids, bonds and conjugates are as described above. The method further can involve co-administering an antiviral agent other than the covalent conjugate. Preferred such antiviral agents are as described above. According to another aspect of the invention, a method is provided for treating a viral infection. The method involves administering to a subject in need of such treatment an amount of a covalent conjugate of a 2′,3′-dideoxycytidine having a nitrogen at the 4-carbon of the pyrimidine ring and a pentose ring and a first fatty acid having 12-26 carbons effective to treat the viral infection, wherein the first fatty acid is conjugated to the nitrogen at the 4-carbon of the pyrimidine ring. Preferred fatty acids, bonds and covalent conjugates are as described above.

According to another aspect of the invention, a method is provided for achieving a therapeutic effect against HIV in HIV infected T cells. The therapeutic effect is enhanced versus that achieved if an equimolar amount of 2′,3′-dideoxycytidine were administered to the subject. The method involves contacting cells with a covalent conjugate of 2′,3′-dideoxycytidine and a first fatty acid. Again, preferred fatty acids, bonds and covalent conjugates are as described above.

According to still another aspect of the invention, a method is provided for achieving a therapeutic effect against a viral infection equivalent to that achieved using a first molar amount of 2′,3′-dideoxycytidine comprising administering to a subject in need of such treatment a conjugate of 2′,3′-dideoxycytidine and a fatty acid in a second molar amount less than the first molar amount. The fatty acids, bonds and preferred covalent conjugates are as described above.

The level of increase is entirely unexpected and unpredictable. Potency was increased by a factor of 50. It is believed that because of the unexpected properties of the conjugates of the invention, dosing of the conjugates of the invention can be reduced by 50%, 60%, 70%, 80% and even 90% or more versus the dosing required when using 2′,3′-dideoxycytidine not conjugated to fatty acid, while achieving the same or even enhanced therapeutic benefit. These and other aspects of the invention are described in greater detail below.

### DETAILED DESCRIPTION OF THE INVENTION

Dideoxycytidine is a nucleoside analog having the following structure:

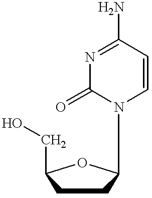

cis-docosahexaenoic acid (DHA) is a naturally occurring fatty acid. It is an unbranched chain fatty acid with six double bonds, all cis. Its structure is as follows:

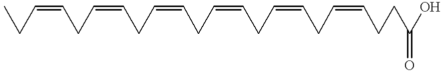

DHA can be isolated, for example, from fish oil or can be chemically synthesized. These methods, however, can generate trans isomers, which are difficult and expensive to separate and which may present safety problems in humans. The preferred method of production is biological synthesis to produce the all cis isomer. The preferred source of DHA is from Martek Biosciences Corporation of Columbia, Md. Martek has a patented system for manufacturing DHA using microalgae which synthesize only a single isomer of DHA, the all cis isomer. Martek's patents include U.S. Pat. Nos. 5,374,657, 5,492,938, 5,407,957 and 5,397,591.

DHA also is present in the milk of lactating women, and Martek's licensee has obtained approval in Europe of DHA as a nutritional supplement for infant formula.

It is known that DHA can be unstable in the presence of oxygen. To stabilize DHA and its conjugates it is important to add anti-oxidants to the material after it is synthesized. One method of stabilization is to make-up the newly synthesized material in the following solution: 100 g neat DHA-ddC plus 100 g of vehicle (100 ml propylene glycol, 70 mg alpha-tocopherol, 5 mg dialaurylthiodipropionic acid, 50 mg ascorbic acid) prepared and held under argon in amber, sealed vials and stored at four degrees centigrade. The following anti-oxidants may also be employed: ascorbic acid, ascorbyl palmitate, dilauryl ascorbate, hydroquinone, butyated hydroxyanisole, sodium meta bisulfite, t-β carotene and α-tocopherol. A heavy metal chelator such as ethylenediamine tetra-acetic acid (EDTA) may also be used.

In one aspect of the invention, cocktails of the ddC-fatty acid conjugate and another antiviral agent can be prepared for administration to subjects having a need for such treatment. One of ordinary skill in the art is familiar with a variety of antiviral agents which are used in the medical arts to treat viral infections. Such agents include nucleoside analogs, nonnucleoside reverse transcriptase inhibitors, protease inhibitors, integrase inhibitors, including the following: Acemannan; Acyclovir; Acyclovir Sodium; Adefovir; Alovudine; Alvircept Sudotox; Amantadine Hydrochloride; Aranotin; Arildone; Atevirdine Mesylate; Avridine; Cidofovir; Cipamfylline; Cytarabine Hydrochloride; Delavirdine Mesylate; Desciclovir; Didanosine; Disoxaril; Edoxudine; Enviradene; Enviroxime; Famciclovir; Famotine Hydrochloride; Fiacitabine; Fialuridine; Fosarilate; Foscarnet Sodium; Fosfonet Sodium; Ganciclovir; Ganciclovir Sodium; Idoxuridine; Indinavir; Kethoxal; Lamivudine; Lobucavir; Memotine Hydrochloride; Methisazone; Nelfinavir; Nevirapine; Penciclovir; Pirodavir; Ribavirin; Rimantadine Hydrochloride; Ritonavir; Saquinavir Mesylate; Somantadine Hydrochloride; Sorivudine; Statolon; Stavudine; Tilorone Hydrochloride; Trifluridine; Valacyclovir Hydrochloride; Vidarabine; Vidarabine Phosphate; Vidarabine Sodium Phosphate; Viroxime; Zalcitabine; Zidovudine; Zinviroxime and integrase inhibitors.

When administered, the formulations of the invention are applied in pharmaceutically acceptable compositions. Such preparations may routinely contain salts, buffering agents, preservatives, compatible carriers, and optionally other therapeutic ingredients. When used in medicine the salts should be pharmaceutically acceptable, but non-pharmaceutically acceptable salts may conveniently be used to prepare pharmaceutically acceptable salts thereof and are not excluded from the scope of the invention. Such pharmacologically and pharmaceutically acceptable salts include, but are not limited to, those prepared from the following acids: hydrochloric, hydrobromic. sulphuric, nitric, phosphoric, maleic, acetic, salicylic, p-toluene sulfonic, tartaric, citric, methane sulfonic, formic, malonic, succinic, naphthalene-2-sulfonic, and benzene sulfonic. Also, pharmaceutically acceptable salts can be prepared as alkaline metal or alkaline earth salts, such as sodium, potassium or calcium salts.

Suitable buffering agents include: acetic acid and a salt (1-2% W/V); citric acid and a salt (1-3% W/V); and phosphoric acid and a salt (0.8-2% W/V).

Suitable preservatives include benzalkonium chloride (0.003-0.03% W/V); chlorobutanol (0.3-0.9% W/V); parabens (0.01-0.25% W/V) and thimerosal (0.004-0.02% W/V).

The active compounds of the present invention may be a pharmaceutical composition having a therapeutically effective amount of a conjugate of the invention optionally included in a pharmaceutically-acceptable carrier. The term “pharmaceutically-acceptable carrier” as used herein means one or more compatible solid or liquid filler, dilutants or encapsulating substances which are suitable for administration to a human or other animal. The term “carrier” denotes an organic or inorganic ingredient, natural or synthetic, with which the active ingredient is combined to facilitate the application. The components of the pharmaceutical compositions are capable of being commingled with the molecules of the present invention, and with each other, in a manner such that there is no interaction which would substantially impair the desired pharmaceutical efficacy.

Compositions suitable for parenteral administration conveniently comprise a sterile preparation of the conjugates of the invention. This preparation may be formulated according to known methods.

The sterile preparation thus may be a sterile solution or suspension in a non-toxic parenterally-acceptable diluent or solvent. In addition, sterile, fixed oils are conventionally employed as a solvent or suspending medium. For this purpose any bland fixed oil may be employed including synthetic mono or di-glycerides. In addition, fatty acids such as oleic acid find use in the preparation of injectables. Carrier formulations suitable for oral, subcutaneous, intravenous, intramuscular, etc. can be found in Remington's Pharmaceutical Sciences, Mack Publishing Company, Easton, Pa.

The invention is used in connection with treating subjects having, suspected of having, developing or suspected of developing a viral infection, particularly a retroviral infection such as HIV. A subject as used herein means humans, primates, horses, cows, pigs, sheep, goats, dogs, cats and rodents.

The conjugates of the invention, when used alone or in cocktails, are administered in effective amounts. An effective amount means that amount necessary to delay the onset of, inhibit the progression of or halt altogether the onset or progression of the viral infection. In particular embodiments, the infection is a retroviral infection, and most particularly an HIV infection. In general, an effective amount will be that amount necessary to inhibit the symptoms or physiological (e.g., immunological or viral) characteristics of the viral infection, any of which otherwise would have occurred in a subject experiencing a viral infection absent the treatment of the invention. Several parameters may be used to assess reduction of viral infection, including inhibited viral replication, a lessened decrease of CD4+ T cell counts, a stabilization of CD4⁺ T cell count or even an increased CD4+ T cell count, and/or an inhibited increase of viral load or even a decreased viral load, for example, as compared to pretreatment patient parameters, untreated patients or, in the case of treatment with cocktails, patients having a viral infection treated with antiviral agents alone (i.e. without the conjugate of the invention). These parameters can be monitored using standard diagnostic procedures including ELISA, polymerase chain reaction (PCR and RT-PCR), and flow cytometry. When administered to a subject, effective amounts will depend, of course, on the particular condition being treated; the severity of the condition; individual patient parameters including age, physical condition, size and weight; concurrent treatment; frequency of treatment; and the mode of administration. These factors are well known to those of ordinary skill in the art and can be addressed with no more than routine experimentation. It is preferred generally that a maximum dose be used, that is, the highest safe dose according to sound medical judgment.

Dosage may be adjusted appropriately to achieve desired drug levels, locally or systemically. Generally, daily oral doses of active compounds will be from about 1 ng/kg per day to 1000 mg/kg per day. It is expected that IV doses in the same range will be effective. In the event that the response in a subject is insufficient at such doses, even higher doses (or effective higher doses by a different, more localized delivery route) may be employed to the extent that patient tolerance permits. Continuous IV dosing over, for example 24 hours or multiple doses per day are contemplated to achieve appropriate systemic levels of compounds. It is believed that dosing can be reduced using the conjugates of the invention by 50%, 60%, 70%, 80%, even 90% or more versus the dosing required when using ddC not conjugated to a fatty acid.

A variety of administration routes are available. The particular mode selected will depend of course, upon the particular drug selected, the severity of the disease state being treated and the dosage required for therapeutic efficacy. The methods of this invention, generally speaking, may be practiced using any mode of administration that is medically acceptable, meaning any mode that produces effective levels of the active compounds without causing clinically unacceptable adverse effects. Such modes of administration include oral, rectal, sublingual, topical, nasal, transdermal or parenteral routes. The term “parenteral” includes subcutaneous, intravenous, intramuscular, or infusion. Intravenous and oral routes are preferred.

The compositions may conveniently be presented in unit dosage form and may be prepared by any of the methods well known in the art of pharmacy. All methods include the step of bringing the conjugates of the invention into association with a carrier which constitutes one or more accessory ingredients. In general, the compositions are prepared by uniformly and intimately bringing the compounds into association with a liquid carrier, a finely divided solid carrier, or both, and then, if necessary, shaping the product.

Compositions suitable for oral administration may be presented as discrete units such as capsules, cachets, tablets, or lozenges, each containing a predetermined amount of the active compound. Other compositions include suspensions in aqueous liquors or non-aqueous liquids such as a syrup, an elixir, or an emulsion.

Other delivery systems can include time-release, delayed release or sustained release delivery systems. Such systems can avoid repeated administrations of the active compounds of the invention, increasing convenience to the subject and the physician. Many types of release delivery systems are available and known to those of ordinary skill in the art. They include polymer based systems such as polylactic and polyglycolic acid, polyanhydrides and polycaprolactone; nonpolymer systems that are lipids including sterols such as cholesterol, cholesterol esters and fatty acids or neutral fats such as mono-, di and triglycerides; hydrogel release systems; silastic systems; peptide based systems; wax coatings, compressed tablets using conventional binders and excipients, partially fused implants and the like. In addition, a pump-based hardware delivery system can be used, some of which are adapted for implantation.

A long-term sustained release implant also may be used. “Long-term” release, as used herein, means that the implant is constructed and arranged to deliver therapeutic levels of the active ingredient for at least 30 days, and preferably 60 days. Long-term sustained release implants are well known to those of ordinary skill in the art and include some of the release systems described above.

### EXAMPLES

Synthesis of an AZT-DHA conjugate

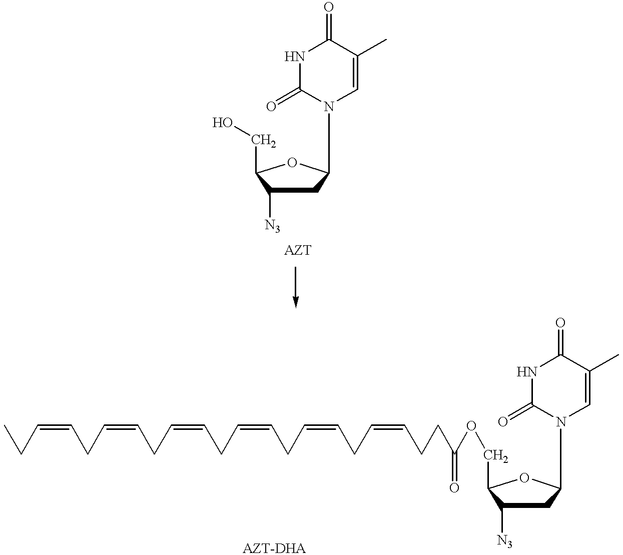

Procedure A:

To a solution of AZT (67 mg, 0.25 mmol) in a 5:1 mixture of CH₂Cl₂ and DMSO (6 ml) were added 4-dimethylaminopyridine (DMAP, 30.5 mg, 0.25 mmol), dicyclohexylcarbodiimide (DCC, 103 mg, 0.5 mmol) and DHA (86 μl, 0.25 mmol) in that order under an Ar atmosphere at room temperature. The reaction mixture was stirred at room temperature for 16 h, then diluted with ether, cooled in the refrigerator (−20° C.) to precipitate the dicyclohexylurea, and filtered through celite. The filtrate was washed with 5% HCl and water followed by brine, dried (Na₂SO₄) and concentrated. The residue was purified by radial chromatography with 3:7 ethyl acetate-hexane as eluent to yield the AZT-DHA as a pale yellow viscous liquid. (45 mg, 31%).

Procedure B:

To a solution of AZT (50 mg, 0.187 mmol) in a 4:1 mixture of CH₂C1₂ and CH₃CN (2.5 ml) were added DMAP (23 mg, 0.187 mmol), DCC (77 mg, 0.374 mmol), and DHA (65 μl, 0.187 mmol) in that order under an Ar atmosphere at room temperature. The reaction mixture was stirred at room temperature for 19 h, then the solvent was removed under reduced pressure, the residue was diluted with ether (15 ml), cooled in the refrigerator (−20° C., 16-18 h), filtered through celite, and the celite pad was washed with ether (3×5 ml). The combined filtrate was dried (Na₂SO₄) and concentrated under reduced pressure. The residue was purified by column chromatography (silica gel) with ethyl acetate-hexane (2:8, 3:7, followed by 1:1) as eluent. The fractions containing the product were concentrated under reduced pressure, diluted with more ether to precipitate traces of dicyclohexylurea and filtered through celite. The filtrate was concentrated under reduced pressure. Further purification of the residue (61 mg) thus obtained by radial chromatography with ethyl acetate-hexane (3:7) as eluent furnished the AZT-DHA analog as a pale yellow liquid (50 mg, 46%).

Procedure C:

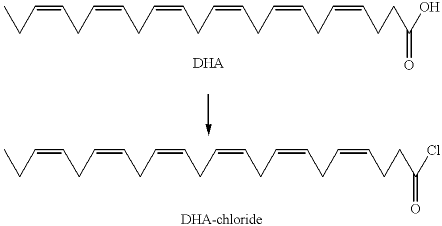

Preparation of DHA-chloride:

To a solution of DHA (265 μl, 0.77 mmol) in CH₂Cl₂ (1 ml) was added thionylchloride (190 μl, 2.6 mmol), at 0° C. under an Ar atmosphere and the reaction mixture was stirred at room temperature for 6 h. Excess thionylchloride was removed by co-evaporation with dry benzene (1.5 ml) under reduced pressure. The resulting acid chloride was dried in high vacuum and subsequently used for the following reaction with further purification.

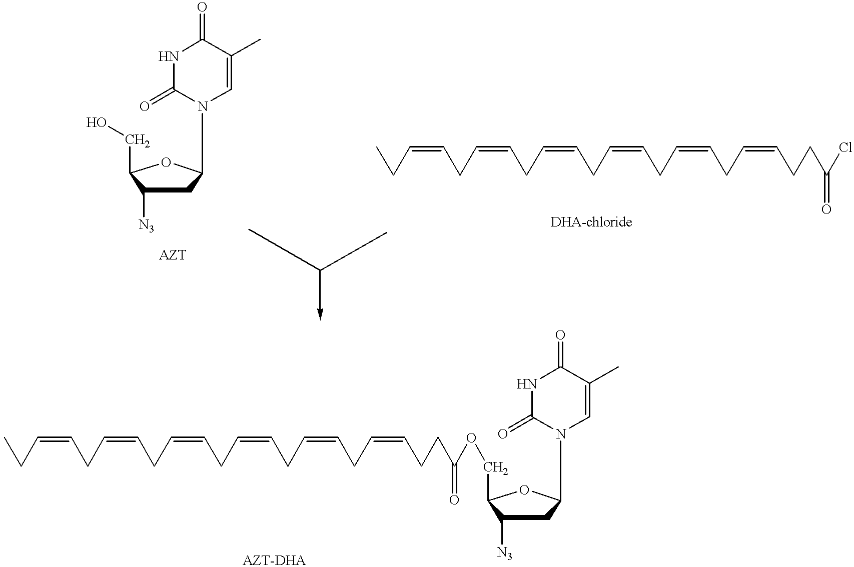

Preparation of AZT-DHA analog:

To a solution of AZT (50 mg, 0.187 mmol) in CH₂Cl₂ (2 ml) and pyridine (50 μl, 0.62 mmol) at 0° C. were added DMAP (23 mg, 0.187 mmol), and DHA-chloride (80 μl, 0.244 mmol) under an Ar atmosphere and the resulting yellow colored solution was stirred at room temperature for 18 h. The reaction mixture was then diluted with more CH₂Cl₂ (30 ml), washed with 5% dil HCl (15 ml) and water (20 ml). The combined aqueous phase was extracted once with CH₂Cl₂ (10 ml). The combined organic phase was washed with brine, dried (Na₂SO₄), and concentrated under reduced pressure. Purification of the dark-colored residue on a short column of basic Al₂O₃ eluting first with 1:1 ethyl acetate-hexane (20 ml) followed by CH₂Cl₂ (30 ml) furnished the AZT-DHA analog as a pale yellow viscous liquid (75 mg, 69%).

NMR analysis of the product was as follows:

¹H NMR (300 MHz, CDCl₃): δ0.97 (t, J=7.6 Hz, 3 H), 1.94 (s, 3 H), 2.08 (apparent quintet, J=7.2 Hz, 2 H), 2.3-2.53 (m, 6 H), 2.79-2.85 (m, 10 H), 4.05-4.09 (m, 1 H), 4.16-4.22 (m, 1 H), 4.36 (d of AB q, J=12.2, 3.8 Hz, 2 H), 5.31 - 5.48 (m, 12 H), 6.1 (t, J=6.4 Hz, 1 H), 7.21 (s, 1 H), 8.63 (brs, 1 H).

¹³C NMR (75 MHz, CDCl₃): δ12.61, 14.22, 20.50, 22.54, 25.48, 25.57, (4 C), 33.92, 37.52, 60.61, 63.29, 81.71, 85.53, 111.27, 126.95, 127.26, 127.77, 127.79, 127.98 (2C), 128.25 (2C), 128.40, 128.53, 129.82, 131.98, 135.19, 150.05, 163.41 and 172.39.

Synthesis of DHA-ddC conjugates

Procedure A: Conjugation of DHA to the pyrimidine ring of ddC

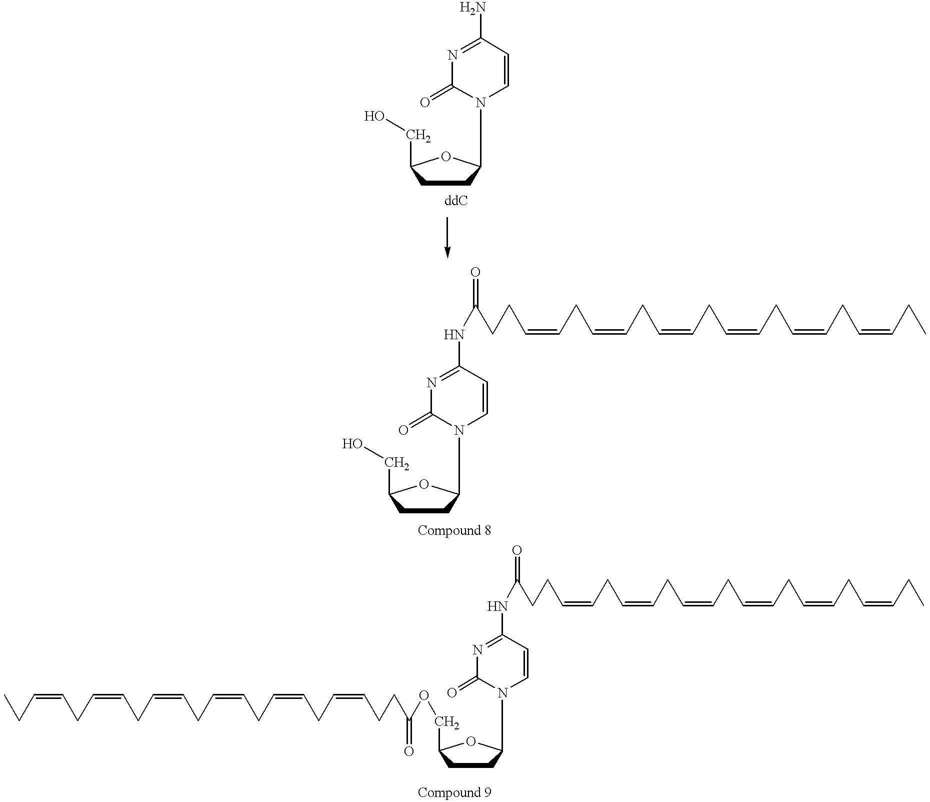

To a solution of DDC (106 mg, 0.5 mmol) in either methylene chloride-dimethylformamide (1:1; 14 ml) or methylene chloride-dimethylsulfoxide (1:1; 14 ml) at ambient temperature under argon were added in sequence 4-dimethylaminopyridine (61.1 mg, 0.5 mmol), 1-hydroxybenzotriazole (67.6 mg, 0.5 mmol), dicyclohexylcarbodiimide (206.3 mg, 1 mmol), and DHA (164.3 mg, 0.5 mmol). The reaction mixture was stirred for 24 h at ambient temperature, diluted with ethyl acetate (40 ml), washed successively with 5% hydrochloric acid, water, saturated aqueous sodium chloride, and dried (sodium sulfate) and concentrated. Radial chromatography (silica gel; ethyl acetate) of the residue afforded 246 mg (47%) of 8 and 140 mg (17%) of 9. DDC-DHA analogs 8 and 9 were stored at −20° C. in ethanol solution (degassed with argon) containing 70 mg α-tocopherol, 5 mg dilauryl dithiopropionate, and 50 mg ascorbic acid per 50 ml.

The results of the NMR analysis of the compounds 8 and 9 were as follows:

8: ¹H NMR (300 MHz; CDCl₃) δ0.96 (t, 3H, J=7.50), 1.88-1.96 (m, 2 H), 2.02-2.15 (m, 2 H), 2.16-2.18 (m, 1 H), 2.39-2.47 (m, 2 H), 2.50-2.54 (m, 1 H), 2.55-2.65 (m, 2 H), 2.78-2.89 (m, 10 H), 3.77 (dd, 1 H, J=3.62, 12.17), 4.05 (dd, 1 H, J=2.21, 12.17), 4.23-4.28 (m, 1 H), 5.26-5.42 (m, 12 H), 6.06 (dd, 1 H, J=2.25, 6.45), 7.47 (d, 1 H, J=7.45), 8.55 (d, 1 H, J=7.45), 9.95 (brs, 1 H).

9: ¹H NMR (300 MHz; CDCl₃) δ0.967 (t, 3 H, J=7.49), 0.969 (t, 3 H, J=7.55), 1.66-1.72 (m, 2 H), 2.02-2.12 (m, 6 H), 2.14-2.17 (m, 1 H), 2.44-2.48 (m, 4 H), 2.52-2.53 (m, 1 H), 2.55-2.65 (m, 2 H), 2.78-2.85 (m, 20 H), 4.33-4.43 (m, 3 H), 5.26-5.45 (m, 24 H), 6.05 (dd, 1 H, J=2.64, 6.55), 7.47 (d, 1 H, J=7.97), 8.15 (d, 1 H, J=7.47), 9.91 (brs, 1 H).

Procedure B: Conjugation of DHA to the pentose ring of ddC

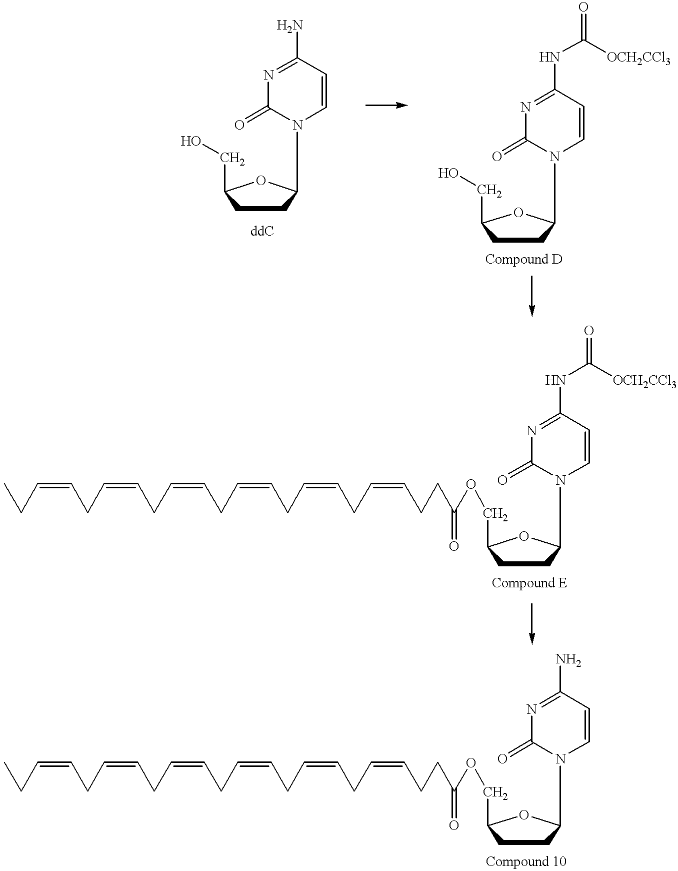

To a solution of DDC (106 mg, 0.5 mmol) in dimethylformamide (10 mil) under argon at ambient temperature were added 4-dimethylaminopyridine (61 mg, 0.5 mmol), pyridine (59 mg, 0.75 mmol), and 2,2,2-trichloroethyl chloroformate (117 mg, 0.55 mmol). The reaction mixture was stirred at ambient temperature for 10 h, diluted with ethyl acetate (30 ml), washed successively with 5% hydrochloric acid, water, saturated aqueous sodium chloride, and dried (sodium sulfate) and concentrated to give 150 mg (79%) of D: ¹H NMR (300 MHz; CDCl₃) δ1.82-1.92 (m, 2 H), 2.08-2.16 (m, 1 H), 2.4-2.52 (m, 1 H), 3.74 (dd, 1 H, J=3.80, 12.05), 4.02 (dd, 1 H, J =2.28, 12.05), 4.18-4.24 (m, 1 H), 4.76 (s, 2 H), 6.02 (dd, 1 H, J=2.28, 6.83), 7.13 (brs, 1 H), 8.58 (d, 1 H, J=7.50).

To a solution of D (150 mg, 0.39 mmol) in methylene chloride (10 ml) under argon at ambient temperature were added in sequence 4-dimethylaminopyridine (47.5 mg, 0.39 mmol), 1-hydroxybenzotriazole (52.6 mg, 0.39 mmol), dicyclohexylcarbodiimide (161 mg, 0.78 mmol), and DHA (128 mg, 0.39 mmol). The reaction mixture was stirred for 10 h at ambient temperature, diluted with ethyl acetate (40 ml), washed successively with 5% hydrochloric acid, water, saturated aqueous sodium chloride, and dried (sodium sulfate) and concentrated. Radial chromatography (silica gel; ethyl acetate) of the residue afforded 250 mg (93%) of E: ¹H NMR (300 MHz; CDCl₃) δ0.90 (t, 3 H, J=7.55), 1.55-1.69 (m, 1 H), 1.92-2.06 (m, 3 H), 2.09-2.16 (m, 1 H), 2.30-2.39 (m, 4 H), 2.43-2.55 (m, 1 H), 2.71-2.91 (m, 10 H), 4.26-4.38 (m, 3 H), 4.76 (s, 2 H), 5.19-5.42 (m, 12 H), 5.97 (dd, 1 H, J=2.64, 6.53), 7.14 (brs, 1 H), 8.11 (d, 1 H, J=7.48).

To a solution of E (250 mg, 0.36 mmol) in tetrahydrofuran (10 ml) under argon was added zinc (500 mg, 2.9 mmol; freshly washed in sequence twice each with 10% hydrochloric acid, water, and tetrahydrofuran) followed by 1M Na₂HPO₄ (2 ml) and the reaction mixture was sonicated in an ultrasonic cleaner for 3 h. The solids were filtered and washed with tetrahydrofuran (20 ml), and the combined filtrates concentrated, Radial chromatography (silica gel; ethanol-methylene chloride) of the residue afforded 130 mg (70%) of 10, which was stored at −20° C. in ethanol solution (degassed with argon) containing 70 mg α-tocopherol, 5 mg dilauryl dithiopropionate, and 50 mg ascorbic acid per 100 ml.

The results of NMR analysis of compound 10 were as follows:

10: ¹H NMR (300 MHz; CDCl₃) δ0.97 (t, 3 H, J=7.54), 1.65-1.72 (m, 1 H), 1.95-2.12 (m, 4 H), 2.35-2.43 (m, 5 H), 2.79-2.89 (m, 10 H), 4.27-4.35 (m, 3 H), 5.26-5.46 (m, 12 H), 5.95 (d, 1 H, J=7.39), 6.02 (m, 1 H), 7.20 (brs, 2 H), 7.69 (d, 1H, J=7.39).

Methods of Use:

Experimental procedures:

Three DHA-ddC (compounds 8, 9, and 10 above) and one DHA-AZT conjugate (the compound described above) were sent to the National Cancer Institute's AIDS antiviral screen to test their anti-HIV activity in vitro. The compounds were provided as solutions in ethanol. The vials containing the conjugates were sealed under argon to prevent oxygen from possibly degrading the conjugates. Instructions were provided to store the vials containing the conjugates at 4° C. and to open the vials immediately before use.

The primary screen used by the NCI for anti-HIV activity utilizes the cytopathicity of HIV-1 for human T4 lymphocytes and the inhibition of such killing by drugs that inhibit viral cytotoxicity. The experimental protocols are described by Weislow et al., (J. Natl. Cancer Inst. 81:577-586, 1989) and Bader (, whose contents are incorporated herein by reference. Briefly, the compounds are diluted first in DMSO or other appropriate solvent, then diluted 1:100 in cell culture medium before preparing serial half log10 dilutions. T4 lymphocytes (CEM line) are added, and after a brief interval HIV-1 (RF strain) is added. Appropriate controls (infected and uninfected cells without compound, and non-infected cells with the compound) are included in the plate format. The cultures are incubated at 37° C. for six days during which time the cells proliferate, the virus reproduces and kills the cells. Cell viability is measured by the ability of cells to convert a colorless tetrazolium salt (XTT) to a highly colored soluble formazan. The intensity of the color is read in a spectrophotometer using an automated system. The wells are also examined microscopically to confirm the protective activity of the compounds.

### RESULTS

The studies below describe the in vitro results obtained with the compounds described above.

1. DHA-ddC at the N.C.I.

The results of the NCI studies are presented for each compound in order of the date the studies were done in Table 1. The mean EC50 for ddC was 1.14×10⁻⁷ M. Fold increase in potency of the DHA-ddC conjugates was calculated by dividing the mean EC50 for ddC (in moles/L) by the EC50 for the individual DHA-ddC conjugates (in moles/L=EC50/MW).

TABLE 1

| | | | | Fold Increase |
|---|---|---|---|---|
| Conjugate | | | | in Potency |
| (# as above) | MW | Date of Test | EC50 | over ddC |
| DHA-ddC 8 | 521 | 7/2/96 | <1.56 × 10⁻⁵ | >3807 |
| | | | μg/ml | |
| | | 7/16/96 | <4.14 × 10⁻⁵ | >3807 |
| | | | μg/ml | |
| DHA-ddC 9 | 831 | 7/2/96 | <1.1 × 10⁻⁵ | >5428 |
| | | | μg/ml | |
| | | 7/16/96 | 4.14 × 10⁻⁵ | 1839 |
| | | | μg/ml | |
| DHA-ddC 10 | 521 | 6/27/96 | 1.02 × 10⁻³ | 57 |
| | | | μg/ml | |
| | | 10/25/96 | <9.22 × 10⁻³ | >6.3 |
| | | | μg/ml | |
| | | 12/3/96 | 7.8 × 10⁻³ | 7.6 |
| | | | μg/ml | |

Conclusions of NCI studies on DHA-ddC:

In the NCI studies of the DHA-ddC conjugates 8 and 9 done on Jul. 2 and 16, 1996, the results show that those covalent conjugates of ddC protected cells against the cytotoxicity of the HIV-1 virus at doses between more than 5,000 fold less than unconjugated ddC and 1800 fold less than unconjugated ddC, i.e., compounds 8 and 9 were between about 1800 and about 5500 fold more potent than ddC itself in protecting against HIV cytotoxicity.

2. DHA-AZT at the N.C.I.

The NCI studies of the DHA-AZT conjugate described above showed that conjugating DHA to AZT did not alter the anti-HIV activity of the conjugate relative to that of the parent drug, AZT. The mean EC50 for AZT alone is 1.77×10⁻⁸ M, while for DHA-AZT it is 2.62×10⁻⁸M.

3. DIA-AZT and DHA-ddC at the Southern Research Institute (SRI)

The results of the experiments performed at SRI are shown in Table 2; the results were expressed in terms of inhibitory concentration

TABLE 2

| | Inhibitory | | | Fold Increase |
|---|---|---|---|---|
| Compound Tested | Concentration | | Control/Expt | in Potency |
| AZT | 0.1 | μM | 0.1/0.1 | 1.0 |
| ddC | 0.3 | μM | 0.38/0.38 | 1.0 |
| DHA | — | | — | — |
| equimolar mixture | 0.06 | μM | 0.1/0.6 | 1.67 |
| of AZT and DHA | | | | |
| equimolar mixture | 0.31 | μM | 0.38/0.31 | 1.23 |
| of ddC and DHA | | | | |
| DHA-ddC | 0.00693 | μM | 0.38/0.00693 | 54.8 |
| compound 8 | | | | |
| DHA-ddC | 0.03 | μM | 0.38/0.03 | 12.7 |
| compound 9 | | | | |
| DHA-ddC | 0.01 | μM | 0.38/0.01 | 38 |
| compound 10 | | | | |
| DHA-AZT | 0.06 | μM | 0.10/0.06 | 1.7 |

The SRI tests showed lesser activity than the NCI ones. The differences between results in the two testing laboratories may have resulted from the compounds having lost some activity on standing between the NCI and the SRI studies. Nevertheless, relative to ddC alone, the three DHA-ddC conjugates showed unexpected increases in activity of between 13 and 55 fold. Relative to AZT alone, DHA conjugation gave no increase in activity.

These in vitro data in human cells establish that conjugating ddC drugs to DHA achieves higher activity (between >5400 fold to 55 to 13 fold) against HIV in human T4 cells. In particular, conjugation of DHA to ddC drugs at the pyrimidine ring of ddC rather than the pentose ring is preferred based upon the unexpected results of the foregoing assays.

The activity of DHA-AZT was not increased relative to AZT alone in the same assays. One possible explanation for this result is that the DHA-AZT conjugate did not have the appropriate stability in tissue culture medium to increase its transport into the T4 cells.

The unexpected findings of increased anti-HIV activity of the DHA-ddC conjugates were not suggested by any previous results. Given the targeting capabilities of DHA, the conjugates will be particularly useful for increasing anti-HIV drug activity in certain cell types and organs (such as the brain), for example to treat AIDS dementia as well as to prevent HIV from migrating out of the brain to re-infect the periphery. Also, the large increase in anti-HIV activity in human T cells in vitro predicts that heretofore untreatable T cell reservoirs of viral infection will become susceptible to anti-viral therapy.

Other aspects of the invention will be clear to the skilled artisan and need not be repeated here. All patents, published patent applications and literature cited herein are incorporated by reference in their entirety.

While the invention has been described with respect to certain embodiments, it should be appreciated that many modifications and changes may be made by those of ordinary skill in the art without departing from the spirit of the invention. It is intended that such modification, changes and equivalents fall within the scope of the following claims.

We claim:

1. A composition of matter comprising
a covalent conjugate of 2′,3′-dideoxycytidine and a first fatty acid having 12-26 carbons, wherein the 2′,3′-dideoxycytidine has a nitrogen at the 4-carbon of a pyrimidine ring and has a pentose ring and wherein the fatty acid is conjugated to the nitrogen at the 4-carbon of the pyrimidine ring.

2. The composition of matter of claim 1, wherein the first fatty acid is an unbranched, naturally occurring fatty acid.

3. The composition of matter of claim 2, wherein the first fatty acid has 14-22 carbons.

4. The composition of matter of claim 1, wherein the first fatty acid is conjugated to 2′,3′-dideoxycytidine via an amide bond between the COOH of the first fatty acid and the NH at the 4-carbon of the pyrimidine ring.

5. The composition of matter of claim 1, wherein the covalent conjugate is

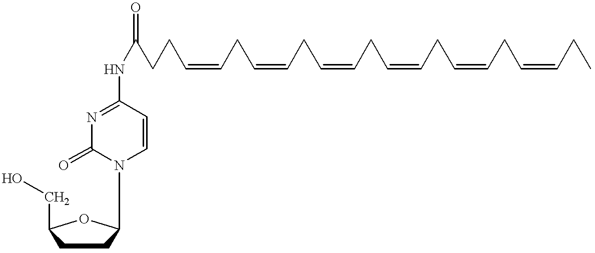

6. The composition of matter of claim 1-5, further comprising a second fatty acid conjugated to the pentose ring.

7. The composition of matter of claim 6, wherein the second fatty acid is an unbranched, naturally occurring fatty acid.

8. The composition of matter of claim 7, wherein the second fatty acid has 14-22 carbons.

9. The composition of matter of claim 6, wherein the second fatty acid is conjugated to 2′,3′-dideoxycytidine via an ester bond between the COOH of the fatty acid and the X-carbon of the pentose ring.

10. The composition of matter of claim 9, wherein the covalent conjugate is

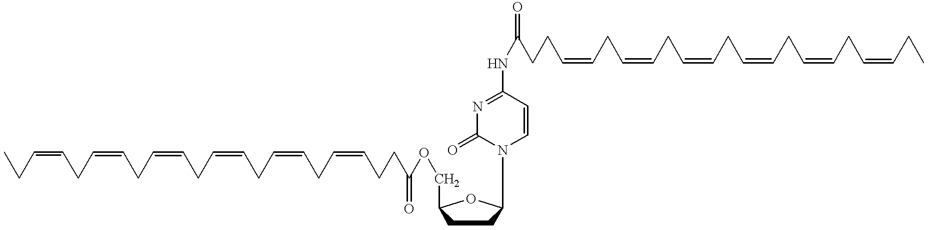

11. A pharmaceutical composition comprising
a covalent conjugate of 2′,3′-dideoxycytidine and a first fatty acid having 12-26 carbons, wherein the 2′,3′-dideoxycytidine has a nitrogen at the 4-carbon of a pyrimidine ring and has a pentose ring and wherein the first fatty acid is conjugated to the nitrogen at the 4-carbon of the pyrimidine ring in an amount effective for treating a viral infection, and
a pharmaceutically acceptable carrier.

12. The pharmaceutical composition of claim 11, wherein the first fatty acid is an unbranched, naturally occurring fatty acid.

13. The pharmaceutical composition of claim 12, wherein the first fatty acid has 16-22 carbons.

14. The pharmaceutical composition of claim 11, wherein the first fatty acid is conjugated to 2′,3′-dideoxycytidine via an amide bond between the COOH of the first fatty acid and the NH at the 4-carbon of the pyrimidine ring.

15. The pharmaceutical composition of claim 11, wherein the covalent conjugate is

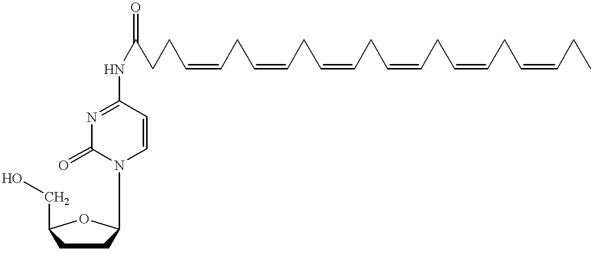

16. The pharmaceutical composition of claim 11-15, further comprising a second fatty acid conjugated to the pentose ring.

17. The pharmaceutical composition of claim 16, wherein -the second fatty acid is an unbranched, naturally occurring fatty acid.

18. The pharmaceutical composition of claim 17, wherein the second fatty acid has 14-22 carbons.

19. The pharmaceutical composition of claim 16, wherein the second fatty acid is conjugated to 2′,3-dideoxycytidine via an ester bond between the COOH of the fatty acid and the X-carbon of the pentose ring.

20. The pharmaceutical composition of claim 16, wherein the covalent conjugate is

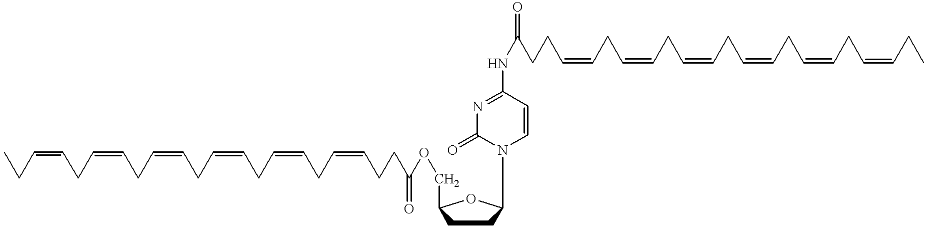

21. The pharmaceutical composition of any of claims 11-15 further comprising an antiviral agent other than the covalent conjugate.

22. The pharmaceutical composition of claim 21 wherein the antiviral agent is selected from the group consisting of nucleoside analogs, non-nucleoside retrovirus inhibitors, protease inhibitors and integrase inhibitors.

23. The pharmaceutical composition of any of claims 11-15, wherein the viral infection is HIV infection.

24. A kit comprising a package housing
a container containing the covalent conjugate of any of claims B1-B8, and also housing instructions for administering to a subject having a viral infection the covalent conjugate.

25. A kit comprising a package housing,
a first container containing the covalent conjugate of any of claims B1-B8, and
a second container containing an anti-viral agent other that the covalent conjugate.

26. A method for treating a non-brain viral infection comprising
administering to a subject in need of such treatment an amount of a covalent conjugate of 2′,3′-dideoxycytidine and a first fatty acid having 12-26 carbons effective to treat the viral infection.

27. The method of claim 26, wherein the first fatty acid is an unbranched, naturally occurring fatty acid.

28. The method of claim 27, wherein the fatty acid has 14-22 carbons.

29. The method of claim 26, wherein the first fatty acid is conjugated to 2′,3′-dideoxycytidine via an amide bond between the COOH of the first fatty acid and the NH at the 4-carbon of the pyrimidine ring.

30. The method of claim 26, wherein the covalent conjugate is

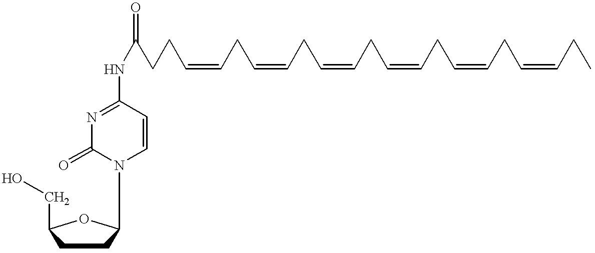

31. The method of claim 26, further comprising a second fatty acid conjugated to the pentose ring.

32. The method of claim 278, wherein the second fatty acid is an unbranched, naturally occurring fatty acid.

33. The method of claim 28, wherein the second fatty acid has 14-22 carbons.

34. The method of claim 29, wherein the second fatty acid is conjugated to 2′,3′-dideoxycytidine via an ester bond between the COOH of the fatty acid and the X-carbon of the pentose ring.

35. The method of claim 30, wherein the covalent conjugate is

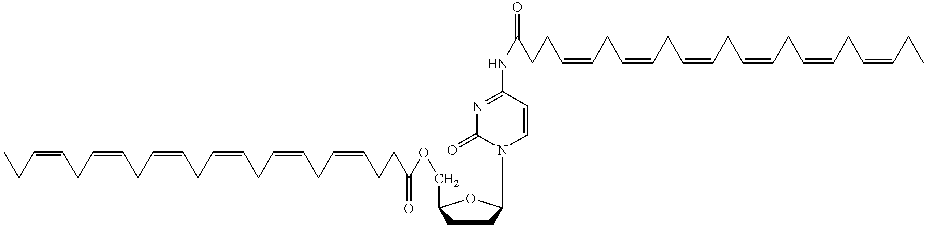

36. The method of claim 26, wherein the first fatty acid is conjugated to 2′,3′-dideoxycytidine via an ester bond between the COOH of the fatty acid and X-carbon of the pentose ring.

37. The method of claim 36, wherein the covalent conjugate is

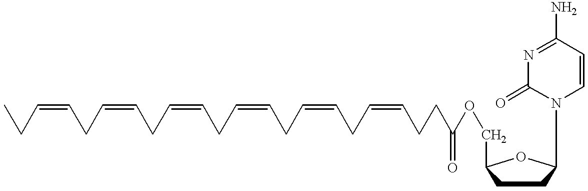

38. The method of any of claim 26, wherein the nucleoside analog is 3′-azido-2′,3′-dideoxythymidine having a pentose ring.

39. The method of claim 38, wherein the first fatty acid is conjugated via an amide bond between the COOH of the fatty acid and the nitrogen of the pentose ring.

40. The method of claim 39, wherein the covalent conjugate is

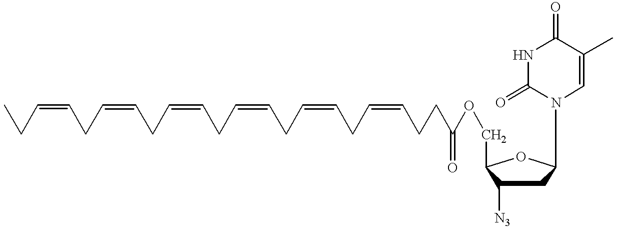

41. The method of any of claims 26-40 further comprising an antiviral agent other than the covalent conjugate.

42. The method of claim 41 wherein the antiviral agent is selected from the group consisting of nucleoside analogs, non-nucleoside retrovirus inhibitors, protease inhibitors and integrase inhibitors.

43. A method for treating a viral infection comprising
administering to a subject in need of such treatment an amount of a covalent conjugate of a 2′,3′-dideoxycytidine having a nitrogen at the 4-carbon of the pyrimidine ring and a pentose ring and a first fatty acid having 12-26 carbons effective to treat the viral infection, wherein the first fatty acid is conjugated to the nitrogen at the 4-carbon of the pyrimidine ring.

44. The method of claim 43, wherein the first fatty acid is an unbranched, naturally occurring fatty acid.

45. The method of claim 44, wherein the first fatty acid has 14-22 carbons.

46. The method of claim 44, wherein the first fatty acid is conjugated to 2′,3′-dideoxycytidine via an amide bond between the COOH of the first fatty acid and the NH at the 4-carbon of the pyrimidine ring.

47. The method of claim 43, wherein the covalent conjugate is

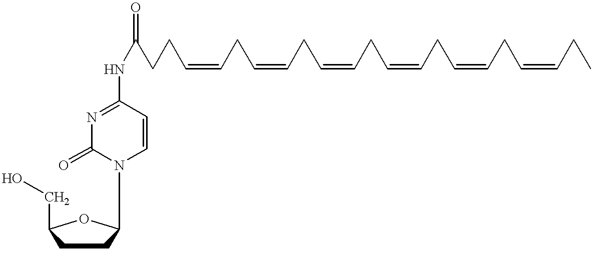

48. The method of claim 43, further comprising a second fatty acid

49. The method of claim 44, wherein the second fatty acid is an unbranched, naturally occurring fatty acid.

50. The method of claim 45, wherein the second fatty acid has 16-22 carbons.

51. The method of claim 46, wherein the second fatty acid is conjugated to 2′,3′-dideoxycytidine via an ester bond between the COOH of the second fatty acid and the X-carbon of the pentose ring.

52. The method of claim 47, wherein the covalent conjugate is

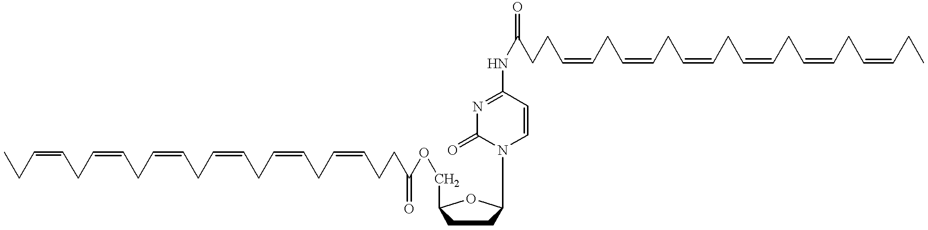

53. A method for achieving a therapeutic effect against HIV in HIV infected T cells that is enhanced versus that achieved when an equimolar amount of 2′,3′-dideoxycytidine is administered, comprising contacting the cells with a covalent conjugate of 2′,3′-dideoxycytidine and a first fatty acid.

54. A method for achieving a therapeutic effect against a viral infection equivalent to that achieved using a first molar amount of 2′,3′-dideoxycytidine comprising
administering to a subject in need of such treatment a conjugate of 2′,3′-dideoxycytidine and a fatty in a second molar amount less than the first molar amount.
